(19) United States
(12) Patent Application Publication
(10) Pub. No.: US 2015/0141890 A1
(43) Pub. Date: May 21, 2015

(54) DYNAMICALLY RESPONSIVE BRACE

(71) Applicant: THE CLEVELAND CLINIC FOUNDATION, Cleveland, OH (US)

(72) Inventors: Jonathan Schaffer, Moreland Hills, OH (US); Salvatore J. Frangiamore, Cleveland, OH (US)

(21) Appl. No.: 14/543,929

(22) Filed: Nov. 18, 2014

Related U.S. Application Data
(60) Provisional application No. 61/905,313, filed on Nov. 18, 2013.

(51) Int. Cl. A61F 5/01

(52) U.S. Cl. CPC A61F 5/0123; A61F 2210/0057
USPC 602/16

(57) ABSTRACT

One aspect of the present disclosure relates to a dynamically responsive brace that includes a flexible member sized and dimensioned to cover a target body region. At least one variable resistance element is associated with the flexible member. The at least one variable resistance element is configured to become exponentially stiffer in response to an applied force that causes deformation of the at least one variable resistance element. Deformation of the at least one variable resistance member restricts pathological motion of the target body region.

### RELATED APPLICATION

This application claims the benefit of U.S. Provisional Patent Application Ser. No. 61/905,313, filed Nov. 18, 2013, the entirety of which is hereby incorporated by reference for all purposes.

### TECHNICAL FIELD

The present disclosure relates generally to medical braces and, more particularly, to a dynamically responsive brace for use in rehabilitation and correction of anatomic abnormalities.

### BACKGROUND

The human knee generally includes an articulating joint between the thigh and calf muscle groups that supports the weight of the body while a person is standing, walking or running. The joint is primarily held together by four small, strong ligaments (e.g., the anterior and posterior cruciate ligaments and the medial and lateral collateral ligaments). The knee is a relatively weak joint and, therefore, knee injuries arising out of cartilage damage, ligament strains, and other causes are relatively commonplace. Knee injuries are particularly likely to occur during physical activities in which the knees are subjected to significant lateral loads.

To help prevent these knee injuries, various types of preventative knee braces have been proposed to help support and reinforce the knee. Such preventative knee braces are rigid and static, and generally include rigid upper and lower members made of metal bars that are connected together by a pair of mechanical hinges, typically made of hard plastic. The upper and lower members are often secured to the leg by a number of straps. The metal bars and plastic hinges used to form the knee brace can be bulky and restrict desired movement of the knee.

### SUMMARY

The present disclosure relates generally to medical braces and, more particularly, to a dynamically responsive brace for use in rehabilitation and correction of anatomic abnormalities.

One aspect of the present disclosure relates to a dynamically responsive brace that includes a flexible member sized and dimensioned to cover a target body region. At least one variable resistance element is associated with the flexible member. The at least one variable resistance element is configured to become exponentially stiffer in response to an applied force that causes deformation of the at least one variable resistance element. Deformation of the at least one variable resistance member restricts pathological motion of the target body region.

Another aspect of the present disclosure relates to a dynamically responsive brace that includes a flexible member sized and dimensioned to cover a target body region. At least one variable resistance element is associated with the flexible member. The at least one variable resistance element is deformed and becomes exponentially stiffer in response to an applied force having a velocity and an acceleration associated therewith. Deformation of the at least one variable resistance member restricts pathological motion of the target body region.

Another aspect of the present disclosure relates to a dynamically responsive knee brace that includes a flexible member sized and dimensioned to cover a knee of a subject. At least one variable resistance element is associated with the flexible member. The at least one variable resistance element is configured to become exponentially stiffer in response to an applied force that causes deformation of the at least one variable resistance element. Deformation of the at least one variable resistance member restricts pathological motion of the knee.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic illustration showing a dynamically responsive brace constructed in accordance with one aspect of the present disclosure;

FIG. 2 is a cross-sectional view taken along Line 2-2 in FIG. 1 showing the dynamically responsive brace without a force applied thereto;

FIG. 3 is a cross-sectional view showing a first force being applied to the dynamically responsive brace in FIG. 2;

FIG. 4 is a cross-sectional view showing a second force, which is greater than the first force in FIG. 3, being applied to the dynamically responsive brace; and

FIG. 5 is a cross-sectional view showing a third force, which is greater than the second force in FIG. 4, being applied to the dynamically responsive brace.

### DETAILED DESCRIPTION

Definitions

Unless defined otherwise, all technical and scientific terms used herein have the same meaning as is commonly understood by one of skill in the art to which the present disclosure pertains.

In the context of the present disclosure, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of the apparatus in use or operation in addition to the orientation depicted in the figures. For example, if the apparatus in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

As used herein, the term “external force” can refer to a force exerted on at least a portion of a target body region of a patient by a source outside of the target body region.

As used herein, the term “internal force” can refer to a force that acts on at least a portion of a target body region of a patient originating from inside the target body region.

Overview

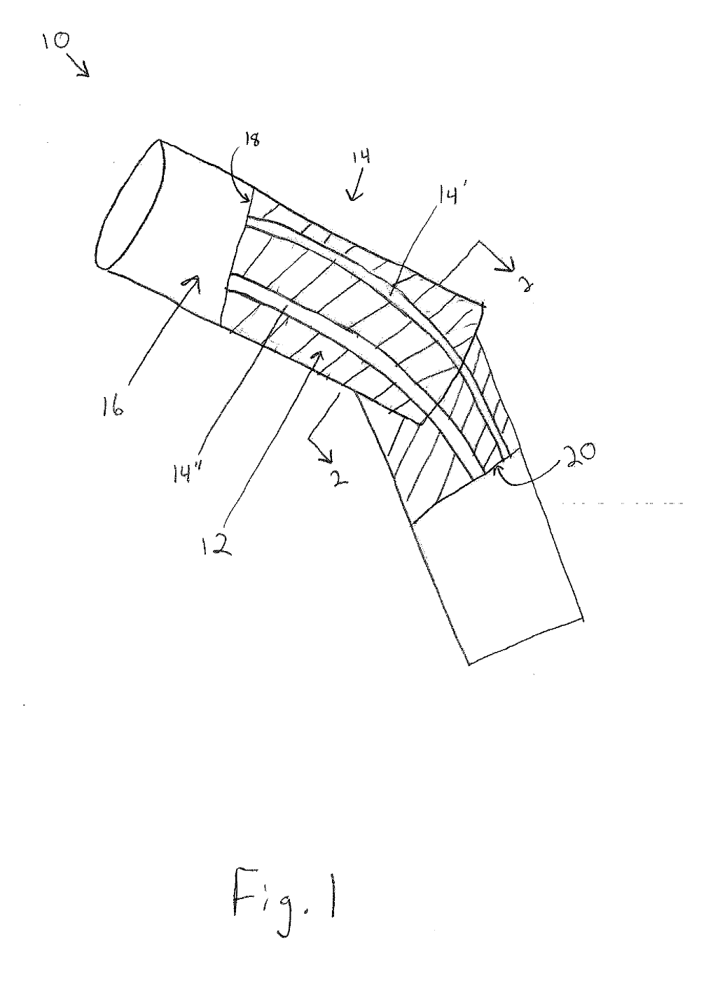

The present disclosure relates generally to medical braces and, more particularly, to a dynamically responsive brace for use in rehabilitation and correction of anatomic abnormalities. As shown in FIG. 1, one aspect of the present disclosure can include a dynamically responsive brace 10 that provides prophylactic and/or therapeutic treatment effects by exponentially stiffening in response to an applied force. Conventional braces typically include rigid metal bars and plastic hinges to support a portion of the patient's body. Such braces tend to be overly restrictive and function-limiting as they restrict motion in a passive, non-dynamic manner. Advantageously, replacing the rigid metal bars and plastic hinges of conventional braces with a dynamically responsive brace 10 that includes active nanocomposite materials (discussed below) creates a more streamlined, lighter, and less bulky alternative to current medical braces. Thus, in some instances, the present disclosure can include a dynamically responsive brace 10 (e.g., in the form of a knee brace) that is physically free of any rigid or semi-rigid support members (e.g., metal bars) and/or hinges (e.g., plastic hinges). Additionally, the active nanocomposite materials impart the dynamically responsive brace 10 with a “selective stiffness”, which allows the dynamically responsive brace to transition between flexible, semi-rigid, and rigid states as needed.

Dynamically Responsive Braces

In one aspect, the dynamically responsive brace 10 can include a flexible member 12 and at least one variable resistance element 14 (VRE) associated with the flexible member. The flexible member 12 can be sized and dimensioned to cover a target body region 16. In some instances, the flexible member 12 can be sized and dimensioned to cover only a target body region, such as only a knee or elbow of a subject. Although the dynamically responsive brace 10 of the present disclosure is primarily described for use in supporting a knee, it will be appreciated that the dynamically responsive brace can be adapted for use with any target body region that is at risk of undesirable deformation or movement. In some instances, the target body region 16 can include any location where two bones come together, such as a hinge joint (e.g., a knee, an elbow, etc.), a ball-and-socket joint (e.g., a shoulder, a hip, etc.), a condyloid joint (e.g., a finger, a toe, etc.), a saddle joint (e.g., a thumb, etc.), a pivot joint (e.g., a neck, etc.), or a gliding joint (e.g., a wrist, an ankle, etc.). In other instances, the target body region 16 can include a non-jointed portion of the patient's body (e.g., a skull, a long bone, etc.). The flexible member 12 can have a variety of shapes and sizes, depending upon the particular target body region 16. In one example, the flexible member 12 can have a sleeve-like shape configured to cover a joint (e.g., a knee). In another example, the flexible member 12 can be configured as a patch to cover a non- jointed target body region 16 (e.g., a portion of a skull following a craniotomy). The flexible member 12 can be made from one or a combination of synthetic materials, such as nylon, elastic, Neoprene, Drytex, HydraCinn® fabric, etc. Although not shown, a strain gauge can be associated with the flexible member 12.

In another aspect, at least one VRE 14 can be associated with the flexible member 12. The VRE(s) 14 can be associated with the flexible member 12 in a variety of ways. For example, the VRE(s) 14 can be integrally formed within the material used to form the flexible member 12, attached to at least a portion of an outer surface of the flexible member (e.g., by stitching, adhesives, etc.), attached to at least a portion of an inner surface of the flexible member, or attached to portions of the inner and outer surfaces of the flexible member. The VRE(s) 14 can be disposed about the flexible member 12 in a variety of shapes and patterns, depending upon the particular target body region 16. For example, first and second VREs 14′ and 14″ can extend from a first end 18 of the flexible member 12 to a second end 20 thereof. The VREs 14 need not be arranged in an identical (e.g., symmetrical) manner on, in, or about the flexible member 12. For instance, a plurality of VREs 14 can be arranged so as to form one or more gradients, each of which having a different force (resistance) profile. In such instances, each of the VREs 14 can have a different maximum deformation threshold so as to create specific resistance pattern. Unlike conventional braces, which only provide static resistance against a single applied force, a brace 10 of the present disclosure which includes one or more gradients can advantageously counteract applied forces from multiple directions.

For application to an MCL sprain, for example, a dynamically responsive brace 10 having first and second gradients can be constructed as follows. The flexible member 12 can be configured to have a tape-like form, such as Kinesio Tape, an ACE bandage, etc. The first gradient can be comprised of one or more VREs 14 to provide medial support, while the second gradient can be comprised of one or more VREs to provide superior/lateral support. The VRE(s) 14 comprising the first gradient can each have a maximum deformation threshold that is higher than the VRE(s) comprising the second gradient. This configuration results in a brace 10 having a more rigid medial portion as compared to the superior/lateral portions, which are less rigid.

In another aspect, the VRE(s) 14 can be made from an active nanocomposite material that allows the VRE(s) to become exponentially stiffer and deform in response to an applied force. In some instances, the VRE(s) 14 can be made according to the layer-by-layer process described in U.S. Pat. No. 7,045,087 (hereinafter, “the '087 patent”). In this instance, the VRE(s) 14 can have a layered configuration and, when prepared according to the process described in the '087 patent, comprise a plurality of aramid nano-fibers. Aramid nano-fibers can impart the VRE(s) 14 with the ability to have a high stiffness (when required) and a low overall weight. As discussed below, the nanocomposite material used to form the VRE(s) 14 can exhibit a maximum deformation threshold at which no further deformation of the VRE(s) occurs, and at which a maximum resistive force is generated by the VRE(s).

In some instances, the VRE(s) 14 can exponentially stiffen in response to an applied external force, an applied internal force, or a combination thereof. In other instances, the VRE(s) 14 can be configured to become exponentially stiffer in response to a velocity and acceleration associated with an applied force. The VRE(s) 14 can provide a resistive force that is substantially opposite in direction and/or magnitude to an applied force. In one example, the VRE(s) 14 can provide a single resistive force that is substantially opposite in direction and/or magnitude to an applied force. In another example, the VRE(s) 14 can provide multiple resistive forces, the sum of which can have a direction and/or magnitude that is/are substantially opposite an applied force. As the VRE(s) 14 deform(s) and become(s) stiffer in response to an applied force, the VRE(s) restrict(s) undesired pathological motion of the target body region 16. The VRE(s) 14 can continue to exponentially stiffen and deform in response to an applied force until a maximum deformation threshold is reached. At the maximum deformation threshold, no further deformation of the VRE(s) 14 occurs, and motion of the target body region 16 is completely restricted or prevented.

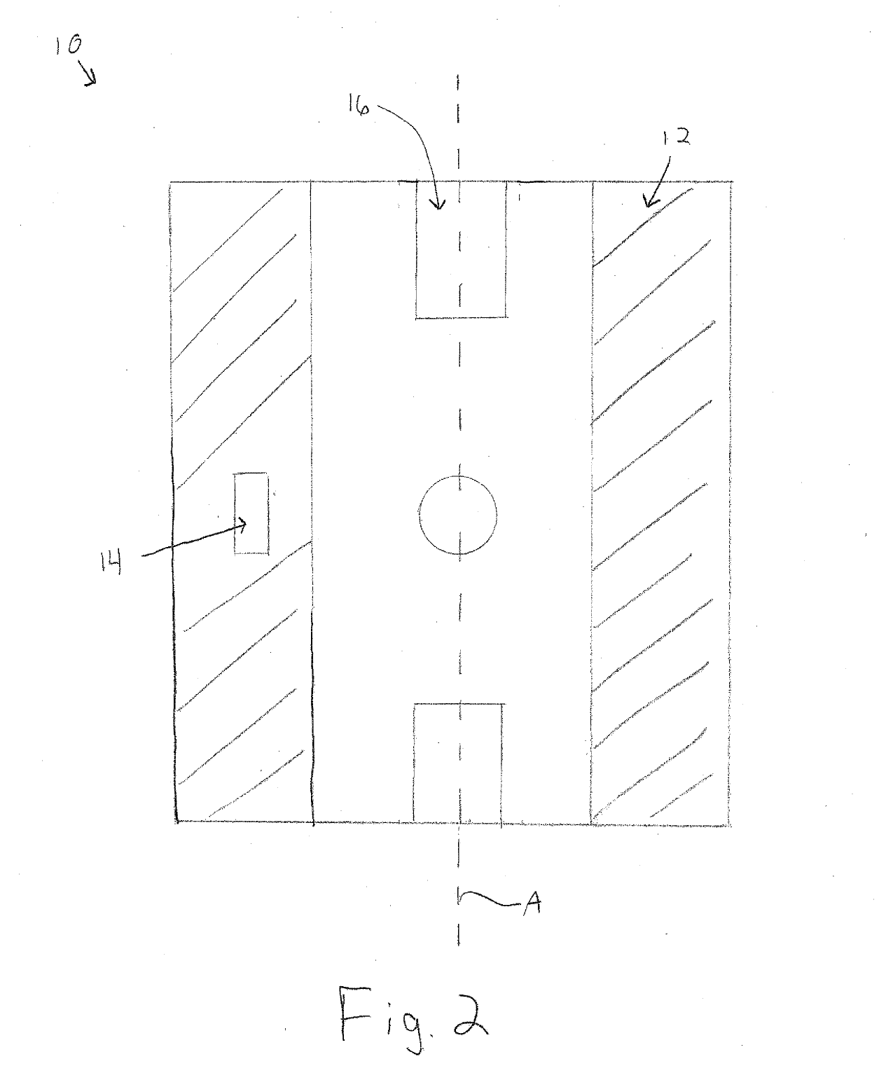

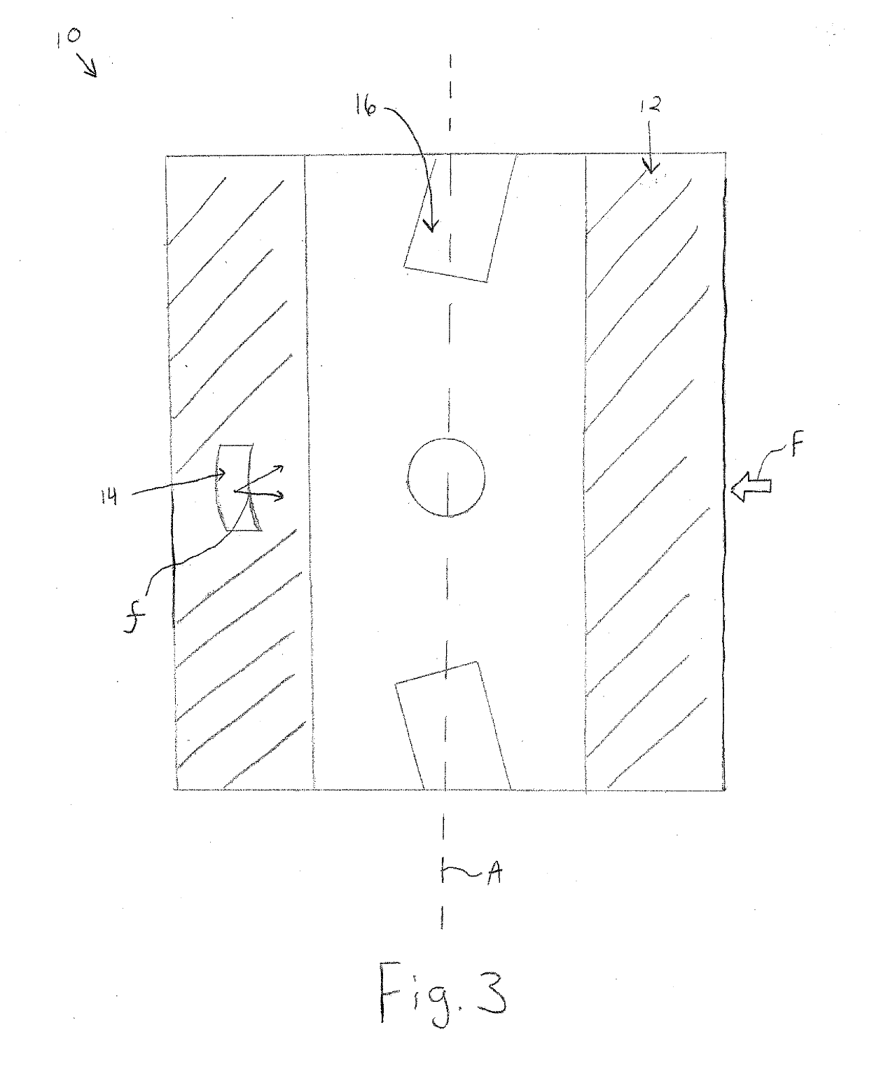

FIGS. 2-5 are a series of cross-sectional views showing the dynamically responsive brace 10 of the present disclosure with a single VRE 14 positioned about a target body region 16 (i.e., the knee) of a subject. FIG. 2 shows the knee and the dynamically responsive brace 10 without any force applied thereto. In this case, the knee is aligned with a normal axis of movement A, in which the femur, fibula, tibia, and patella are in normal vertical alignment with one another. Also in this case, the VRE 14 does not exhibit any substantial deformation in the absence of an applied force. When an external applied force F (FIG. 3) is applied to a lateral aspect of the knee, the knee is undesirably displaced (e.g., laterally) from the normal axis of movement A. At the same time, the VRE 14 begins to deform in response to the applied force F. Stiffening of the VRE 14 also generates one or more resistive forces f, which have a direction substantially opposite the direction of the applied force F. Consequently, the resistive force(s) f can slow and/or stop deviation of the knee from the normal axis of movement A.

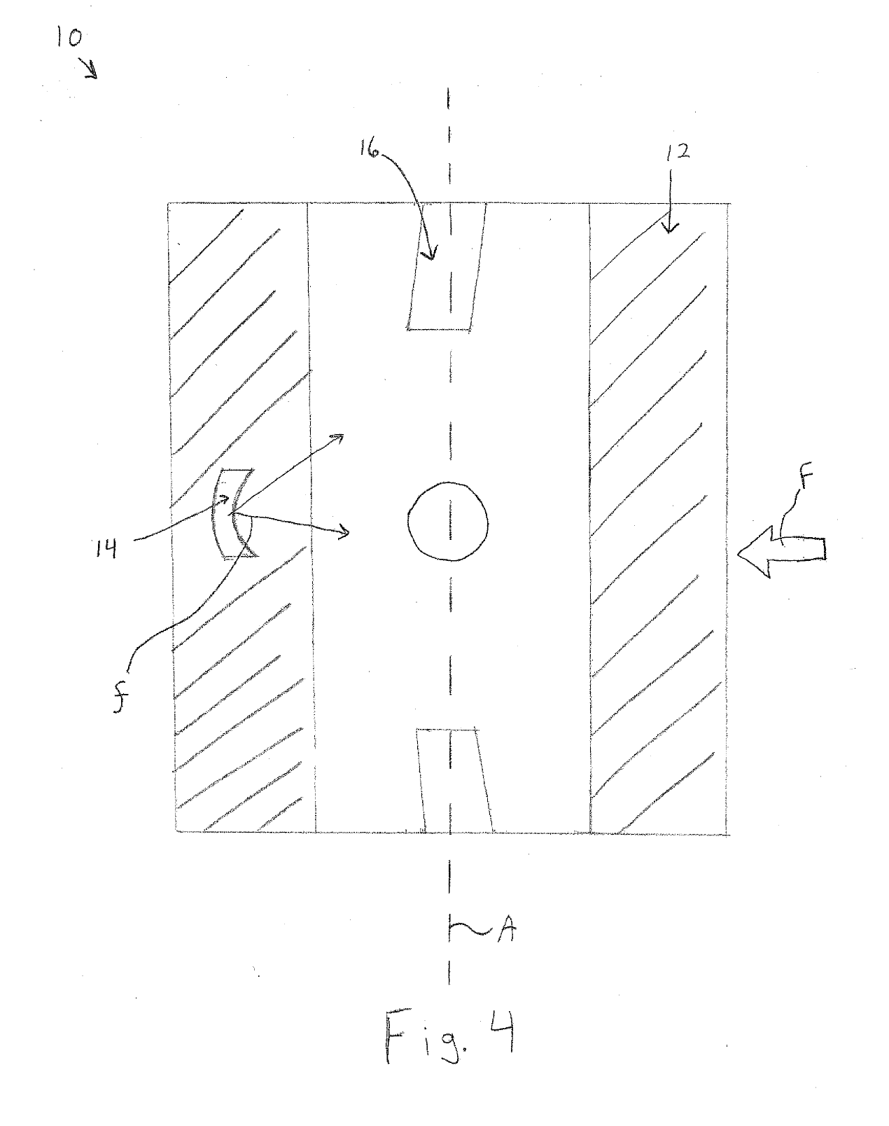

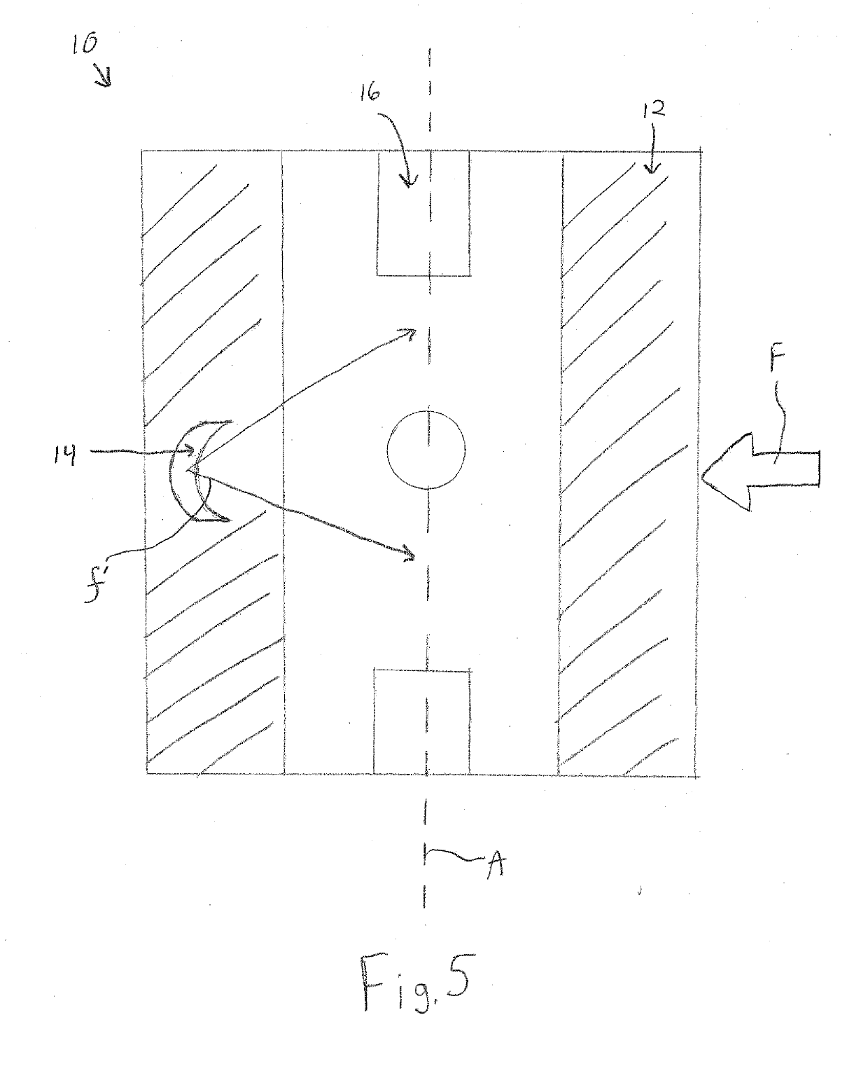

As shown in FIG. 4, the deformation and stiffness of the VRE 14 increases as the magnitude of the applied force F increases. The exponential increase in stiffness of the VRE 14 also causes the resistive force(s) f exerted by the VRE to increase. As this occurs, deviation of the knee from the normal axis of movement A is further reduced. The VRE 14 continues to deform and exponentially stiffen in response to the applied force F until the maximum deformation threshold is reached (FIG. 5). At this point, the VRE 14 does not deform further and exerts a maximum resistive force f′ that has a direction and magnitude substantially equal to the direction and magnitude of the applied force F. Consequently, undesired pathological motion (e.g., lateral motion) of the knee from the normal axis of movement A is completely restricted or prevented.

From the above description of the present disclosure, those skilled in the art will perceive improvements, changes and modifications. For example, it will be appreciated that the brace 10 can be constructed in a similar or identical manner as a conventional knee brace, such as a prophylactic brace, a functional brace, a rehabilitative brace, or an unloader brace. Such improvements, changes, and modifications are within the skill of those in the art and are intended to be covered by the appended claims. All patents, patent applications, and publication cited herein are incorporated by reference in their entirety.

The following is claimed:

1. A dynamically responsive brace comprising:
a flexible member sized and dimensioned to cover a target body region; and
at least one variable resistance element associated with the flexible member;
wherein the at least one variable resistance element is configured to become exponentially stiffer in response to an applied force that causes deformation of the at least one variable resistance element;
wherein deformation of the at least one variable resistance member restricts pathological motion of the target body region.

2. The dynamically responsive brace of claim 1, wherein a strain gauge is associated with the flexible member.

3. The dynamically responsive brace of claim 1, wherein the at least one variable resistance element is associated with at least a portion of an outer surface of the flexible member, at least a portion of an inner surface of the flexible member, or both.

4. The dynamically responsive brace of claim 1, wherein the at least one variable resistance element exhibits a maximum deformation threshold at which no further deformation occurs and motion of the target body region is completely restricted.

5. The dynamically responsive brace of claim 1, wherein the force applied to the at least one variable resistance element is an external force, an internal force, or a combination thereof.

6. A dynamically responsive brace comprising:
a flexible member sized and dimensioned to cover a target body region; and
at least one variable resistance element associated with the flexible member;
wherein the at least one variable resistance element is deformed and becomes exponentially stiffer in response to an applied force having a velocity and an acceleration associated therewith;
wherein deformation of the at least one variable resistance member restricts pathological motion of the target body region.

7. The dynamically responsive brace of claim 6, wherein a strain gauge is associated with the flexible member.

8. The dynamically responsive brace of claim 6, wherein the at least one variable resistance element is associated with at least a portion of an outer surface of the flexible member, at least a portion of an inner surface of the flexible member, or both.

9. The dynamically responsive brace of claim 6, wherein the at least one variable resistance element exhibits a maximum deformation threshold at which no further deformation occurs and motion of the target body region is completely restricted.

10. The dynamically responsive brace of claim 1, wherein the force applied to the at least one variable resistance element is an external force, an internal force, or a combination thereof.

11. A dynamically responsive knee brace comprising:
a flexible member sized and dimensioned to cover a knee of a subject; and
at least one variable resistance element associated with the flexible member;
wherein the at least one variable resistance element is configured to become exponentially stiffer in response to an applied force that causes deformation of the at least one variable resistance element;
wherein deformation of the at least one variable resistance member restricts pathological motion of the knee.

12. The dynamically responsive knee brace of claim 11, wherein a strain gauge is associated with the flexible member.

13. The dynamically responsive knee brace of claim 11, wherein the at least one variable resistance element is associated with at least a portion of an outer surface of the flexible member, at least a portion of an inner surface of the flexible member, or both.

14. The dynamically responsive knee brace of claim 11, wherein the at least one variable resistance element exhibits a maximum deformation threshold at which no further deformation occurs and lateral motion of the knee is completely restricted.

15. The dynamically responsive knee brace of claim 11, wherein the force applied to the at least one variable resistance element is an external force, an internal force, or a combination thereof.

16. The dynamically responsive knee brace of claim 11, wherein the knee brace is physically free of any metal bars or hinges.
